(19) United States
(12) Patent Application Publication
(10) Pub. No.: US 2024/0382136 A1
(43) Pub. Date: Nov. 21, 2024

(54) IMAGING OF SEIZURE SOURCES USING BIOPHYSICALLY-CONSTRAINED DEEP NEURAL NETWORKS

(71) Applicant: CARNEGIE MELLON UNIVERSITY, Pittsburgh, PA (US)

(72) Inventors: Rui SUN, Pittsburgh, PA (US); Abbas SOHRABPOUR, Pittsburgh, PA (US); Bin He, Pittsburgh, PA (US)

(73) Assignee: CARNEGIE MELLON UNIVERSITY, Pittsburgh, PA (US)

(21) Appl. No.: 18/751,633

(22) Filed: Jun. 24, 2024

Related U.S. Application Data
(63) Continuation-in-part of application No. 17/315,691, filed on May 10, 2021, now Pat. No. 12,016,634.
(60) Provisional application No. 63/022,876, filed on May 11, 2020.

(51) Int. Cl. A61B 5/384; A61B 5/00; A61B 5/372

(52) U.S. Cl. CPC A61B 5/384; A61B 5/372; A61B 5/725; A61B 5/7264

(57) ABSTRACT

Disclosed herein is a novel deep learning-based source imaging framework for imaging ictal oscillations from high-density electrophysiological recordings in drug-resistant focal epilepsy patients. A neural mass model producing ictal oscillations was used to generate synthetic training data having spatio-temporal-spectra features indicative of ictal oscillations. The synthetic training data was then used to train the deep learning-based source imaging framework to image and localize brain source patches exhibiting ictal oscillations, based on an input of EEG data.

### RELATED APPLICATIONS

This application is a continuation-in-part application of pending U.S. patent application Ser. No. 17/315,691 (filed May 10, 2021), which claims the benefit of U.S. Provisional Patent Application No. 63/022,876 (filed May 11, 2020). The contents of these applications are incorporated herein in their entireties.

### GOVERNMENT INTEREST

This invention was made with government support under grants from the National Institutes of Health, Nos. NS127849 and NS096761. The government has certain rights in this invention.

### BACKGROUND

Neural information processing is encoded by rhythmic oscillations. Noninvasive imaging of the network dynamics of such neural rhythms is of significance for elucidating the mechanism of brain function and aiding in the clinical management of brain disorders. Epilepsy is a common neurological disorder affecting around 70 million patients worldwide, one third of whom cannot be managed by medication alone. For focal drug resistant epilepsy (DRE), in which seizures are originated in a focal region of the brain, surgery to remove the epileptogenic zone (EZ) is the most effective treatment option. Accurately identifying the epileptogenic tissue is of great importance to the diagnosis and treatment planning for these patients. Patients selected for surgical treatment usually undergo extensive intracranial EEG (iEEG) monitoring (including stero-EEG or electrocorticography) to determine the epileptogenic tissue from invasive ictal recordings, which is a close approximation of the EZ. However, iEEG sometimes is limited by its spatial coverage, as well as the risk and discomfort associated with the invasive procedure. There is thus a clinical need for noninvasive imaging techniques with high spatiotemporal resolution, to identify and localize seizure generating tissues with high accuracy.

Electroencephalography and magnetoencephalography (E/MEG) are noninvasive techniques that can record neural activities with high temporal resolution. However, limited spatial information can be inferred from the scalp measurements because of the low signal to noise ratio (SNR), small sensor counts, and volume conduction effects. Electrophysiological source imaging (ESI) techniques have been developed to boost the spatial resolution of E/MEG by estimating the underlying brain dynamics from E/MEG recordings. ESI involves solving the forward problem and the inverse problem. The forward problem models the source space as a distribution of current dipoles, and its mapping relationship to the sensor space as a linear projection. Because the number of scalp measurements is much smaller than the number of current dipoles, a regularization procedure is conventionally used to solve the underdetermined inverse problem. However, the performance of the ESI solution is limited by the representation power of the regularization term.

Deep learning-based source imaging approaches solve the ESI problem under a different framework. Instead of being formulated explicitly as a regularization term, source dynamics can be implicitly embedded in the training data and learned by the neural networks through the training process. It is, however, challenging to acquire enough simultaneously recorded iEEG and scalp data to train such a model. Computational models can serve as a powerful alternative to introduce the source dynamics into training data if the source models are realistic enough for source imaging tasks. DL-based source imaging framework (DeepSIF) is an ESI framework utilizing dynamic brain network models as the source model in the forward problem, and the deep neural networks (DNN) to solve the inverse problem. Different source models can be selected based on source signal properties and the modalities of the measurements, to generate various types of source-sensor signal pairs as the training data. DeepSIF has been successfully applied to image transient activities such as evoked potentials or interictal spike activities from EEG or MEG signals, demonstrating powerful generalization capabilities cross subjects and modalities.

On the other hand, the oscillatory activities in EEG signals are fundamental for inferring physiological or pathological information about specific brain states or disorders. One example is the occurrence of strong rhythmic patterns during seizures; The origin of these patterns is a major part of determining the EZ. Ictal ESI could be more informative than interictal ESI results, however, ictal EEG recordings are usually contaminated by large artifacts. Although independent component analysis (ICA) can remove some artifacts, ictal oscillations still have low SNR due to non-ictal brain rhythms, making the analysis of these patterns challenging. To overcome these issues, several approaches have been proposed, such as transforming the data into the frequency domain or averaging the signal at the peak of the ictal oscillations to increase the SNR of the ictal recordings, albeit at the expense of temporal resolution. Averaging the EEG over time, while effective in improving SNR, can reduce the spatial specificity of the ESI results. Thus, it is important to develop a robust spatiotemporal imaging method that can model the ictal neural oscillations when analyzing the ictal EEG data.

### SUMMARY OF THE INVENTION

An accurate noninvasive ictal imaging technique from scalp electrophysiological recordings, including EEG and MEG, could offer a clear hypothesis regarding the EZ area, which would subsequently facilitate iEEG implantation and may ultimately lead to the complete noninvasive source localization of seizure generating tissues, potentially leading to improved patient outcomes.

As a general ESI framework, DeepSIF has been shown to provide excellent spatiotemporal ESI results on non-oscillatory activities at low SNR. Disclosed herein is a method for expanding the capability of DeepSIF to imaging oscillatory activities, specifically, the ictal oscillations. FIG. 1 is a schematic illustrating the method. Ictal spatial and temporal source models generate synthetic training data for the DNN. The trained network was then utilized to image and localize seizure generating tissues from scalp recorded high density ictal EEG (or MEG) in focal DRE patients. The resulting images are compared to iEEG defined seizure onset zones (SOZ) and/or resection volumes. DeepSIF successfully images ictal activities with a high degree of temporal correlation with the scalp recordings and high spatial precision that agreed with the clinical ground truth. These results suggest that DeepSIF has great potential in advancing the noninvasive imaging of ictal activities in patients with focal epilepsy, which could provide valuable insights to guide clinical decisions and improve treatment outcomes.

Given the potential benefits in epilepsy treatment, extending DeepSIF to imaging rhythmic activities provides a great advantage for the imaging of ictal activities. As it has demonstrated promise in localizing the sources of brain activity with high accuracy and robustness, by extending DeepSIF for ictal activities, it is possible to create a more comprehensive and reliable methodology for identifying EZ noninvasively.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic illustrating the method of the present invention.

FIG. 2 is a schematic illustration of components of a system implementing the methods of the present invention.

FIG. 3 shows examples of ictal oscillation patterns generated by the modified Jansen-Rit model for each signal type.

FIG. 4 is a graph showing signal type classification examples for various parameters of the neural mass model.

FIG. 5 shows various examples of 2 seconds of waveform when varying parameters of the neural mass model.

FIG. 6 shows results on test dataset with temporal dynamics varying in time, including spatial specificity, spatial sensitivity, localization error, and temporal correlation. Included are imaging examples showing source locations and waveforms (1 second) of the simulated and reconstructed sources.

FIG. 7 shows results on test dataset with temporal dynamics varying in space, including spatial specificity, spatial sensitivity, localization error and temporal correlation. Included are imaging example showing two dynamics presented in one patch denoted by red and yellow.

FIG. 8 shows the imaging performance for the two datasets at different SNR.

FIG. 9 shows examples of ictal-imaging results along with the surgical resection outcome and iEEG defined seizure onset zones.

FIG. 10 shows examples of ictal and spike imaging results.

FIG. 11 shows examples of ictal-imaging results along with the surgical resection outcome and iEEG defined seizure onset zones.

FIG. 12 shows schematic diagrams of a system with the present invention for recording EEG (or MEG), processing recorded signals, training a deep learning neural network using biophysical models, and displaying source imaging results for guiding neuroscience research or clinical applications.

### DETAILED DESCRIPTION

Disclosed herein is a novel electrophysiological source imaging method using biophysically-constrained deep neural networks to robustly and accurately localize and image spatiotemporal distribution of seizure sources from EEG (or MEG) ictal recordings. The method has been rigorously validated in a cohort of drug resistant focal epilepsy patients by comparing noninvasive source imaging results with clinical ground truth based on intracranial EEG defined seizure onset zone and successful surgical resection outcome. The DL-based ictal source imaging approach demonstrates superior performance over existing methods.

DL-based ESI methods are capable of implicitly learning the source distributions through data instead of explicitly formulating the regularization terms to constrain the solution space, which means more complex source models can be incorporated into the solution to achieve a more accurate and robust source estimate. DeepSIF as a DL-based ESI method, has proven to be effective for imaging transient activities such as interictal spikes or evoked potentials in a group of subjects. It is a modular framework consisting of a forward source model, using neural mass models, to generate realistic synthetic training data, and an inverse neural network model to perform the ESI task based on the information in the training data. The two components are closely connected though the training data as high-quality training data needs to be fed into the neural network for an optimal training result. However, they are also independent as the detailed implementations and assumptions in the source model are implicitly embedded into the training data and the complexity of the source model will not affect the optimization process for the inverse module. Thus, different forward source models and inverse network structures for DeepSIF can be adopted based on the specific task to achieve optimal results.

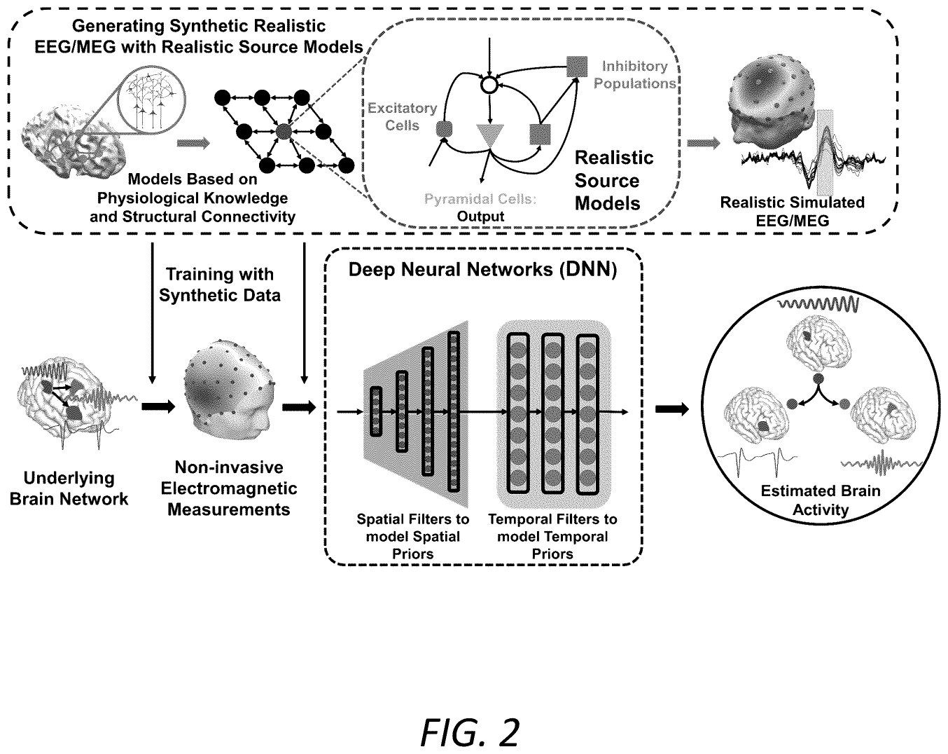

FIG. 2 is a schematic illustration of the methods for EEG/MEG source imaging using deep learning neural networks. As illustrated in FIG. 2, signals generated by a realistic source model are used as the underlying brain activity to generate the synthetic noninvasive EEG or MEG signals. The synthetic scalp and the corresponding brain signals are used as input to train a neural network which, in various embodiments of the invention, may be a deep neural network (DNN). With proper training, the neural network returns source distributions corresponding to an EEG or MEG input that represent spatio-temporal distributions of brain activity and functional networks.

Similar to the EEG or MEG illustrated in FIG. 2, signals generated by a realistic source model can be used as the underlying brain activity to generate the synthetic iEEG signals. The synthetic iEEG and the corresponding brain signals can be used as input to train a neural network which, in various embodiments of the invention, may be a deep neural network (DNN). With proper training, the neural network returns source distributions corresponding to an iEEG input that represent spatio-temporal distributions of brain activity and functional networks.

When using iEEG to perform ESI estimation, while the recordings are invasive, the source localization allows the localization and imaging of brain signal origins from recorded iEEG signals, which can help resolve the limited coverage of iEEG recordings.

To generate the training data for the ictal DeepSIF model, a modified Jansen-Rit model was adopted for generating training data with various spatio-temporal-spectra features. Multiple computational models have been proposed to describe and analyze the dynamics of seizures. Concepts of increased tissue excitability, impaired dendritic inhibition and coupling interactions have been incorporated into models at various scales to provide insights regarding the biophysical mechanisms of seizure initiation, propagation and termination at micro-to-macro scales. Developing a seizure model that can fully describe the seizure process is an active research area. Therefore, certain simplifications were made when selecting and constructing the source model for DeepSIF, with the primary focus being on reproducing realistic phenomena observed in the recorded signals, rather than elaborating on the underlying mechanisms. This approach prioritizes the practical application and performance of DeepSIF in real-world situations.

First, the neural mass model (NMM) must generate a wide range of ictal dynamics. Models have been proposed to model and explain certain types of ictal oscillations. The modified Jansen-Rit model has demonstrated the ability to model different types of ictal oscillations, with a wide range of parameter sets for each type of signal. A large number of parameter sets were explored and included in the training data, making it a suitable model to build a training dataset with high varieties. Second, the parameters were manually modified for the transition between ictal signal types. Some models can autonomously transit between dynamical states by setting the NMM system close to the bifurcation and providing random fluctuations as the input. However, this limits the possible parameter selections, thus the possible waveform dynamics in the training data. As a trade-off, transitions between states were modeled by manually changing the NMM parameters to generate different ictal signal types. Similarly, the spatial dynamic variations are also modeled by manually changing the parameters. Only a short time segment of seizure was considered in the source model. As a seizure could last from a couple seconds to couple minutes, it is challenging to include all the possible temporal variation patterns and spatial propagation patterns in the training data through the whole course. The problem was simplified by using the assumption that the ictal source is piece-wise stationary, which means in a short segment of time, the source is confined in a focal region. Then, the size and shape of the seizure source can be properly defined by a patch, and the spatial dynamic variations can be introduced by manually modifying the signal types in the center segments and neighboring segments of the source patch. These assumptions and simplifications provide a valid trade-off between the realism and the implementation feasibility of the source model, and it is suitable for the source imaging tasks.

Generating realistic datasets has been one of the bottlenecks for the advancement of DL-based ESI methods. The development of the spatiotemporal ictal source model provides a training dataset with enough spatio-temporal-spectra features, which is a critical step in providing an accurate ictal imaging result. The DeepSIF model was trained on a generic head model, and it was rigorously validated in computer simulations and in a cohort of focal DRE patients. The trained DeepSIF model demonstrates superior robustness and generalizability on various test conditions, and it can be successfully applied to different patients with certain spatiotemporal robustness, which provides a high level of efficiency as single trained model can be directly applied at multiple test conditions.

### Methods-Ictal Oscillation Simulations Using Modified Jansen-Rit Model

The neural mass model (NMM) is a “mean-field” computational model that describes the collective dynamics and interactions among groups of neurons and has been widely used as the source models for iEEG/EEG/MEG measurements. Different types of NMMs have been proposed to model epilepsy-related activities. Jansen-Rit and its modified version are among the most popular NMMs with physiologically meaningful parameters. The Jansen-Rit model contains three neural subpopulations, the pyramidal neurons, the excitatory, and the inhibitory feedback interneurons. Fast inhibitory interneurons are added in the modified Jansen-Rit model to model different types of ictal oscillations. Each subpopulation is characterized by a dynamic impulse response function h (t) that transforms the pre-synaptic information (the average action potential firing rate) to the post-synaptic information (the mean membrane potential), and a static nonlinear activation function that transforms the mean membrane potential to an average firing rate.

The impulse response function is defined as: h(t)=Qqte^{-qt}, t>0, where Q is the average synaptic gain and it is denoted as A, B, G for excitatory, slow inhibitory and fast inhibitory population, respectively. q is the average membrane time constant modeling the time delay and it is denoted as a, b, g for the excitatory, slow inhibitory and fast inhibitory populations, respectively. It has been shown that by modifying the synaptic gain parameters in the impulse response function, 6 types of signals can be simulated with the modified Jansen-Rit model: Normal activity (Type 1), sporadic spikes (Type 2), sustained discharge of spikes (Type 3), rhythmic activity (Type 4), low voltage rapid activity (Type 5) and quasi-sinusoidal activity (Type 6).

The impact of the gain parameter A, B, G and time delay constant a, b, g in the impulse response function on the signal dynamics were explored. The gain parameter A was set to 5.5 (to reduce the parameter space to explore) as it can provide all 6 signal dynamics when B and G vary. To further reduce the parameter search space, simulations were first performed for different B and G parameters. Generated signals were then classified into 6 groups based on line-length, major frequency, and baseline. B, G pairs generating normal activities were removed from further simulations. Then for each pair of valid B, G, a grid search of a, b, g was performed. B was varied from 2 to 60 mV with a resolution of 2 mV, G was varied from 2 to 30 mV with a resolution of 2 mV, a was varied from 0.05 to 0.15 ms-1 with a resolution of 0.01 ms-1, b was varied from 0.025 to 0.075 ms-1 with a resolution of 0.005 ms-1, g was varied from 0.2 to 1.6 ms-1 with a resolution of 0.2 ms-1. Five seconds of signal were generated with step size of 0.5 second for each parameter set. A total of around 210,000 sets of simulations were performed. Note that this is not an exhaustive exploration of the full parameter space of the modified Jansen-Rit model. The goal is to identify enough variations of the NMM parameter sets for the training data generation. Signal types 2-6 were possible ictal dynamics and were included in the training data. To ensure a uniformed representation of different types of ictal signals, parameter sets for each signal type were resampled to 10,000. A total of 50,000 parameter sets were identified. 40,000 sets were used as the candidate parameter sets to generate the ictal training data, and 10,000 sets were used for the ictal testing data.

### Model Training and Evaluation

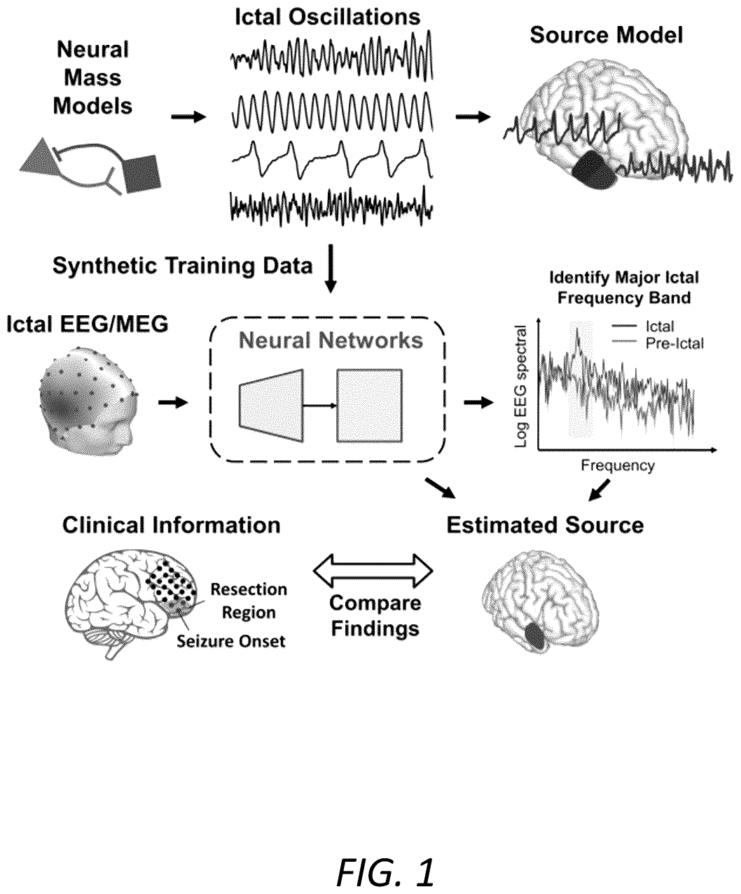

After identifying the parameter sets for a single NMM to generate the ictal oscillations, they were combined with the spatial-temporal source model to simulate the EEG signals during the ictal periods. A template magnetic resonance imaging (MRI) (fsaverage5) was used to generate the head model and its cortical surface was segmented into 994 regions with each region modeled by one modified Jansen-Rit model. The spatial model is a region growing method, where the center segment was chosen randomly to determine the source location, and the source patch was created by randomly grouping the neighboring segments with the center segment. Each source patch consisted of two types of spatiotemporal dynamics. In the first type, the entire patch shared the same temporal waveform. In the second type, the source patch was separated into the center segment group and the neighboring segment group (See FIG. 1, top right). Two groups had different temporal waveforms to simulate the phase differences or change of dynamics after the signal propagates to neighboring regions. The size of the center segment group was randomly selected, and the segments included were chosen using the region-growing method. The remaining cortical segments in the source patch comprised the neighboring segment group. The NMM parameter for the source patch (entire patch, or the center/neighboring segment groups) was selected randomly from the parameter sets obtained from the previous step. Thus, source patches with different sizes, shapes, locations, and temporal dynamics were generated. The 76-channel electrode layout based on a 10-10 montage was used as the EEG electrode configuration by projecting the template EEG cap onto the scalp surface of fsaverage5. The lead-field matrix was calculated using the 3-shell boundary element method (BEM) model with openMEEG in Brainstorm with default settings. In total, a training dataset containing 1,240,512 brain-scalp signal samples (620 million spatial topographical distributions) were generated at signal to noise ratios (SNR) of 5, 10, 15, or 20 dB.

The network consisted of a spatial module to pre-filter the EEG signal and a temporal module to model the temporal dynamics. Both the source and sensor space signals were scaled by their maximum absolute value to have a maximum or minimum of 1 or −1. During training, the loss function was the mean square error loss (MSE) between the model output and the ground truth source activity. The Adam optimizer was used for the training with a weight decay of 1e⁻⁶. The learning rate was 3e-4 and the batch size was 64. The whole network was implemented in PyTorch and trained on one NVIDIA Tesla V100 GPU.

The source patches in the test dataset were separately generated following the same protocol as the training data with different NMM parameters. Two test datasets were created with different temporal dynamics. In the first test dataset, the entire patch shared the same temporal waveform, but the ictal signal types changed within one test sample. In each test sample, the switch in the waveform dynamics, happened at a random time point. In the second test dataset, similar to the training set, there were different temporal waveforms in one source patch. Each dataset contains single source data with 23,856 samples at 5-20 dB SNR levels. Samples in these two datasets were used as the input for the trained model.

Otsu's thresholding technique was used to identify the boundary of the imaged source distribution. Modified spatial sensitivity and specificity were used to evaluate the extent estimation accuracy. The localization error (LE) is defined as the average of the distance from the estimated source to the ground truth and the distance from the ground truth to the estimated source. The correlation value is defined as the maximum Pearson correlation between reconstructed and simulated waveforms. One test sample consists of multiple cortical regions and the LE and correlation for one test sample is the mean values for all regions in the reconstructed source.

### Ictal Oscillation Simulations

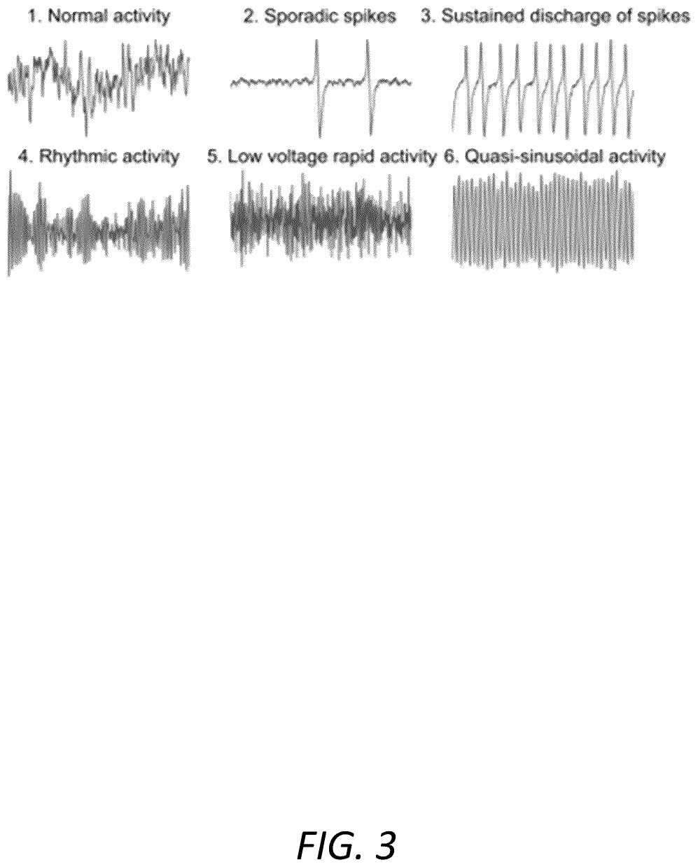

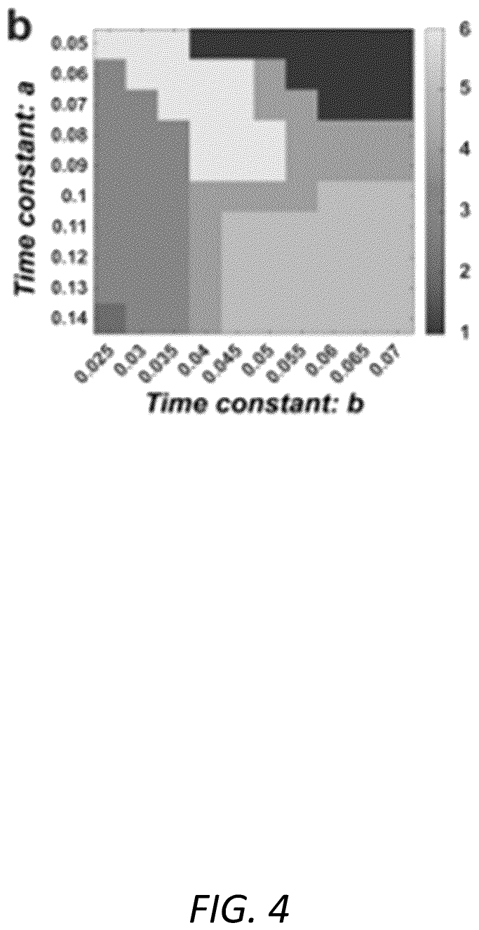

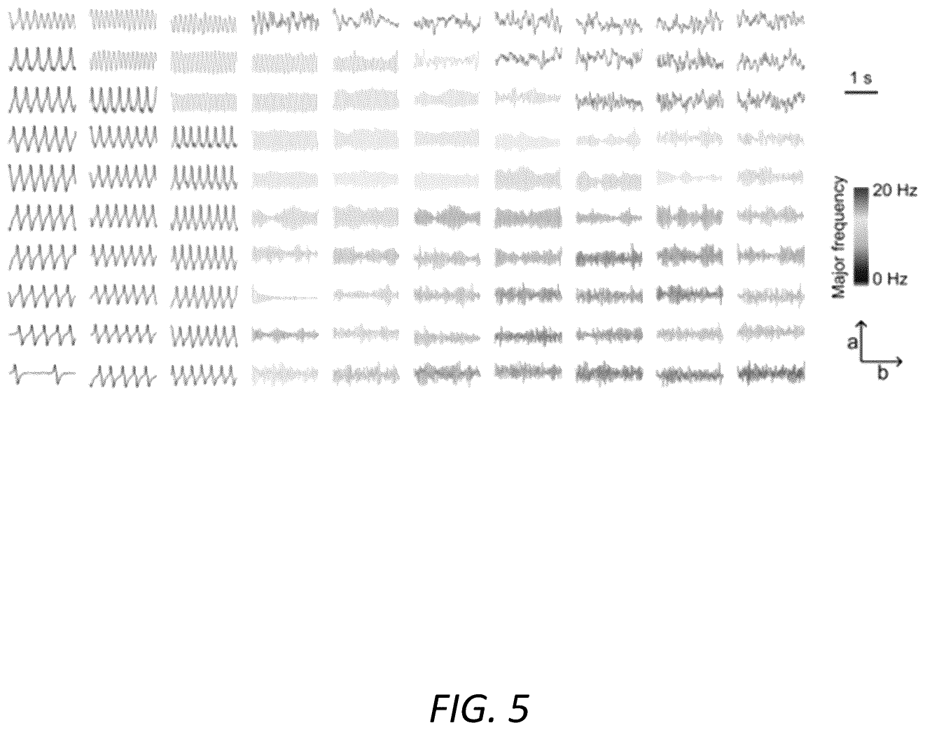

The modified Jansen-Rit model, a neural mass model (NMM), was adopted as the temporal model for the ictal signal. FIG. 3 shows the typical waveforms in each of the 6 signal groups: normal activity, sporadic spikes, sustained discharge of spikes, rhythmic activity, low voltage rapid activity and quasi-sinusoidal activity. A typical example of the NMM simulation result (at A=5.5 mv, B=14 mv, G=4 mv, g−0.5 ms⁻¹) is shown in FIG. 4. The color represents the signal type transition when varying the time constant a and b. With these sets of NMM parameters, NMM can generate five types of signals typical in ictal oscillations with different temporal and spectra features. FIG. 5 shows the waveforms generated by the corresponding a, b parameters in FIG. 4. Although the signals share similar temporal features within each signal type/category, their major frequency gradually changes with different time constant values, providing more variations for the training dataset.

### DeepSIF Performance in Computer Simulations

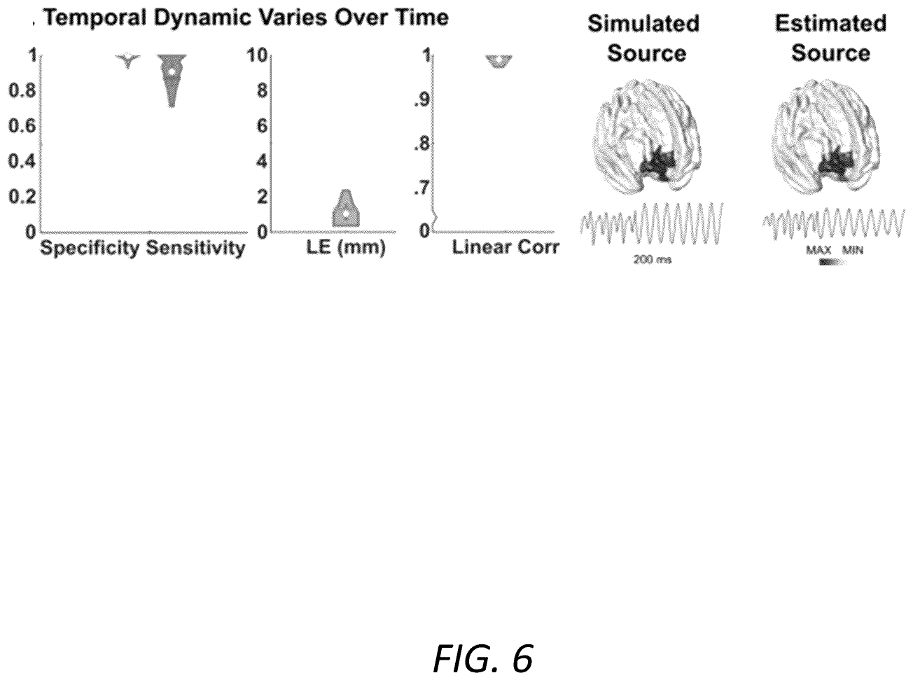

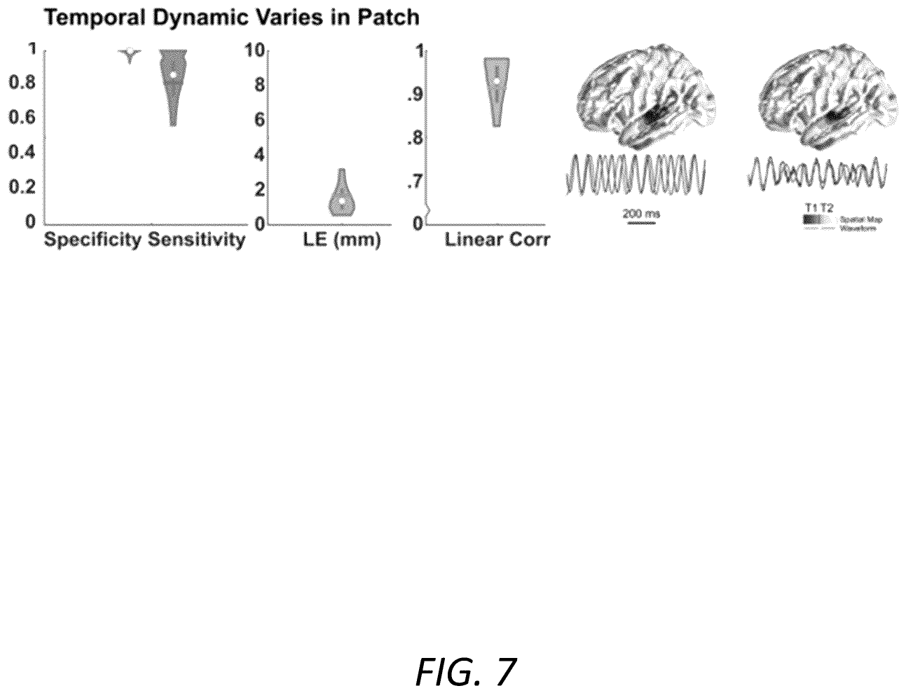

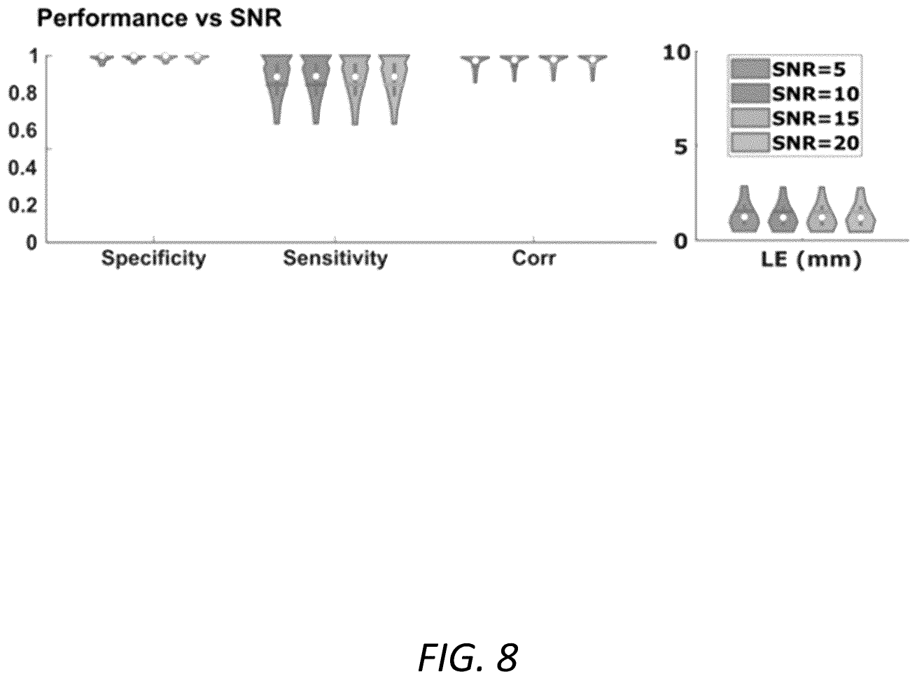

A DeepSIF model with skip connections and recurrent layers was trained with synthetic ictal data, comprising of source-sensor paired signal activities generated by the NMM. The ability for DeepSIF to detect temporal dynamics variations in time and in space was then evaluated on two test datasets and is shown in FIG. 6. High spatial specificity and sensitivity can be achieved for both test datasets. When the oscillation pattern varies over time, DeepSIF can identify the change and provide the correct temporal estimation with high linear correlation with the simulated signal (0.98±0.04). Note that in the training data, ictal type remains constant over time for one training sample. However, due to the presence of various ictal types in the training dataset through different samples, the DNN still has the capability to accurately image the source even when the oscillation pattern varies. It is more challenging when two different dynamics are presented in one patch. As illustrated in the example in FIG. 7, a single patch contains two sinusoidal oscillations with a phase difference, and DeepSIF can provide distinct temporal estimates for each source. At around 200 ms, the source signal phases are reversed, resulting in less accurate estimations from DeepSIF due to signal cancellation in the sensor space. However, the recurrent structure allows DeepSIF to utilize information from previous time points to provide more reliable estimations for ambiguous time points. The temporal correlation of the simulated and estimated signal remains high (0.92±0.07) for this test dataset, despite the temporal inferences inside the source patch. This highlights the model's ability to adapt and maintain accuracy even in challenging situations. The aggregated results for the two test datasets at different SNR are described in FIG. 8, demonstrating consistent performance across all SNR levels. The simulation study demonstrated that DeepSIF can deliver accurate and robust source estimates for sources exhibiting a wide range of spatiotemporal patterns, emphasizing its reliability and adaptability in varying conditions.

### Patient Data Analysis

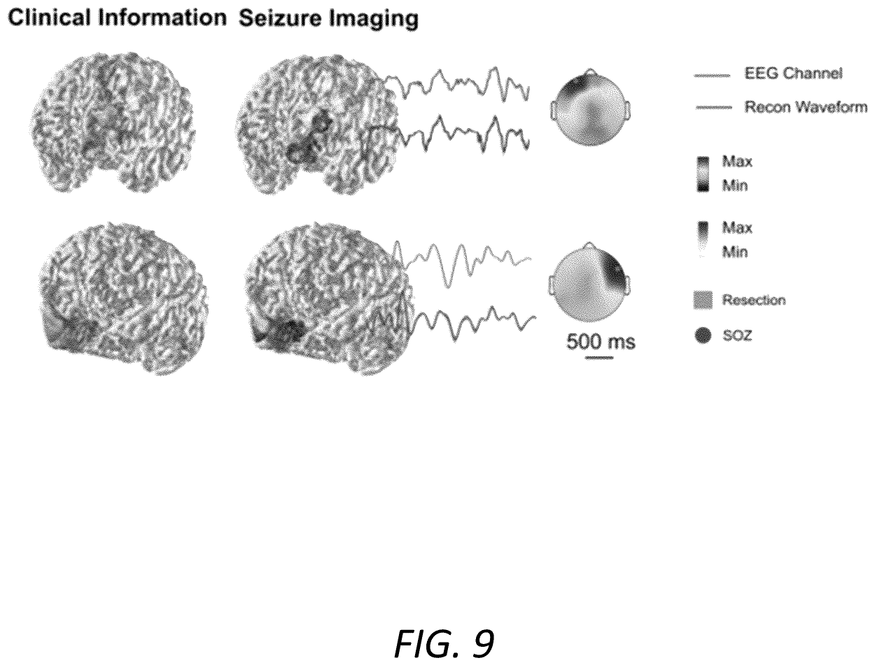

The DeepSIF performance for imaging real ictal signals was evaluated in a cohort of 33 focal DRE patients. FIG. 9 presents examples of ictal imaging, showcasing the excellent performance of DeepSIF when compared to surgical resection outcomes and iEEG-defined seizure onset zone (SOZ). A high spatial specificity is obtained (0.96±0.90), indicating that the noninvasive DeepSIF source imaging results have minimum spurious activities extending outside the epileptogenic region, as also evident by the low spatial dispersion value (3.80±5.74 mm). The average distance from the SOZ electrode to the reconstruction area is 10.89±10.14 mm for all patients and 8.03±9.01 mm for seizure-free patients. Furthermore, the reconstructed waveform exhibits a high temporal correlation (0.81±0.14) with the EEG signals, demonstrating DeepSIF's capability in reconstructing the temporal dynamics from EEG traces.

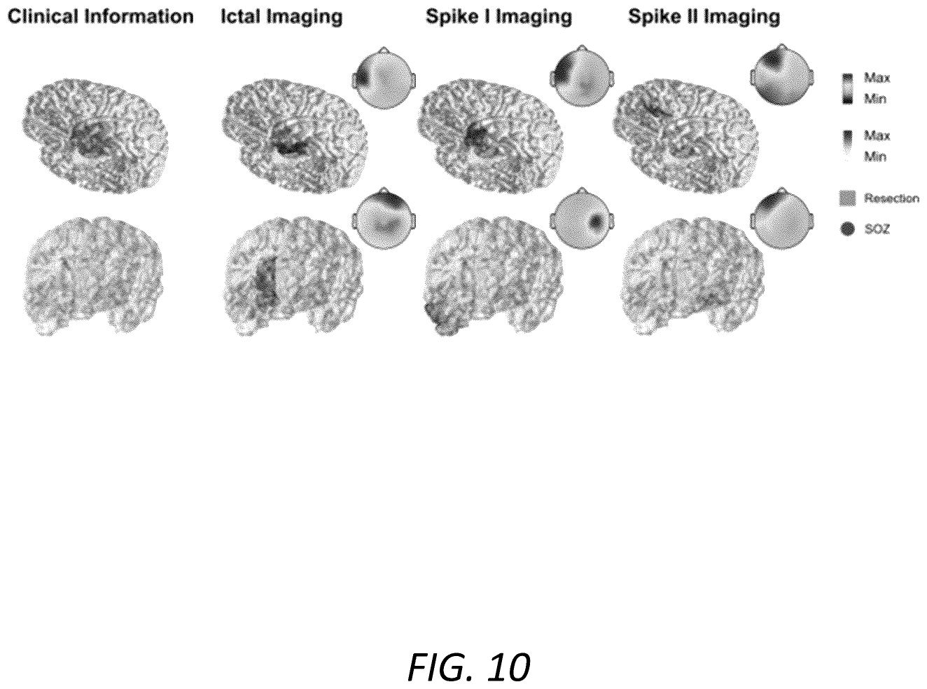

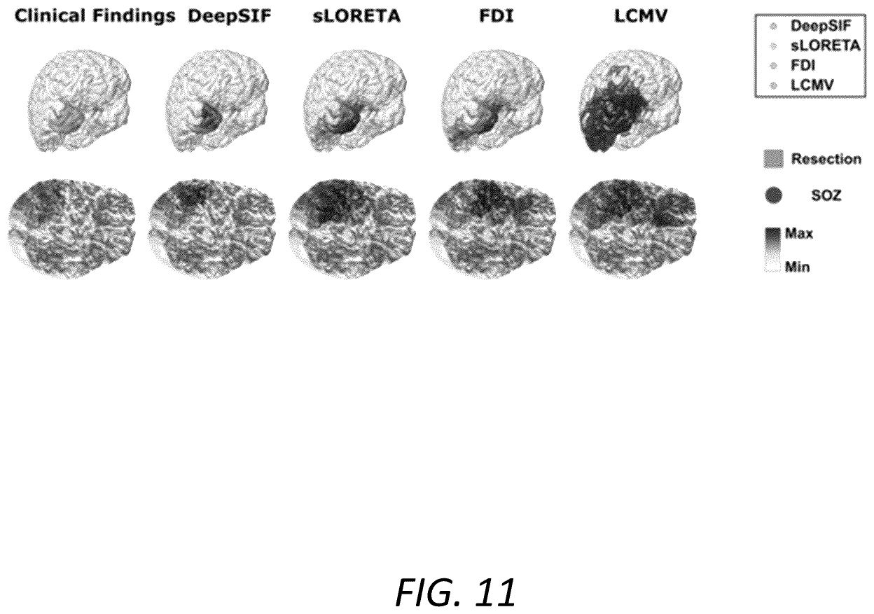

When comparing the results of ictal and spike imaging, statistically significant differences can be observed for all metrics except spatial dispersion. The spatial dispersion for spike imaging exhibits a bimodal distribution (Hartigan's dip test, p<0.01) and can be divided into seizure-free (SZ-free) and non-seizure-free (non-SZ-free) groups. The spatial dispersion value is significantly lower for ictal imaging compared to spike imaging in the non-SZ-free group. This difference in significance can also be observed in the non-SZ-free group for spatial sensitivity and specificity. In general, the primary distinction between ictal imaging and spike imaging lies in the non-SZ-free group. This is predominantly due to the fact that many non-SZ-free patients experience multiple types of spikes, several of which are contralateral to the clinical ground truth. Even when the source can be lateralized before spike analysis, it remains challenging to differentiate between different spike groups, as demonstrated in the example shown in FIG. 10. On the other hand, seizure onset locations tend to be more consistent, with fewer contralateral or discordant seizures. This consistency highlights the value of ictal imaging in providing a more accurate representation of seizure activity, particularly for non-SZ-free patients. FIG. 11 shows the comparison of DeepSIF with other benchmark ESI methods: time domain standardized low resolution brain electromagnetic tomography (sLORETA), frequency domain imaging with sLORETA (FDI), and time domain linearly constrained minimum variance beamformer (LCMV). DeepSIF demonstrated superior imaging performance, achieving a SOZ LE of 16.94±9.08 mm, which is significantly better when compared to the benchmark methods. While LCMV and sLORETA can provide high sensitivity, their low specificity hampers their ability to accurately identify true epileptic regions. DeepSIF offers a balanced sensitivity and specificity value, allowing for a more accurate representation of the source extent without being excessively focal or overly diffused. DeepSIF offers a balanced sensitivity and specificity value, allowing for a more accurate representation of the source extent without being excessively focal or overly diffused. The geometric mean of the sensitivity and specificity can reflect the overall performance for estimating the resection regions, and it was 0.62±0.22 (DeepSIF), 0.60±0.15 (sLORETA), 0.54±0.16 (FDI), and 0.47±0.17 (LCMV). Precision and recall are also important metrics to evaluate the overlap between two areas. The sensitivity is the same as the recall. The precision value compared to the resection area and the F1-score (the harmonic mean of the precision and recall) demonstrate that DeepSIF outperformed all other benchmark algorithms in terms of source extent imaging.

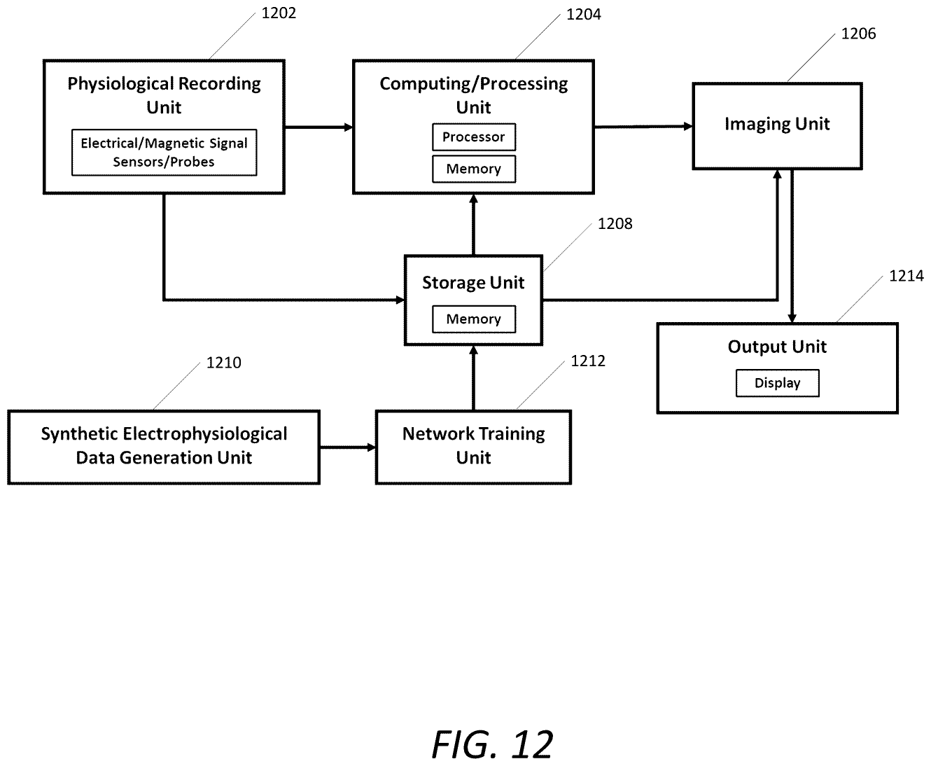

A block diagram of an apparatus implementing one embodiment of the present invention is shown in FIG. 12. A Physiological Recording Unit 1202 is used to collect electric or magnetic signals using a plurality of sensors and records electromagnetic signals at the scalp or within the brain/over brain surface from sensors of EEG, MEG, iEEG or a combination of EEG, MEG, and iEEG, a combination of EEG and MEG, a combination of EEG and iEEG, or a combination of MEG and iEEG. The recordings are passed to the Storage Unit for long-term keeping and to be sent to the Computing/Processing Unit for further analysis. The Storage Unit 1208 saves electromagnetic recordings, the deep neural network architecture, weights, and data as well as all processed data in between and at final stages. Synthetic Electrophysiological Data Generation Unit 1210 generates surface EEG (or MEG) data using the neural field model consisting of a number of NMM models and a head volume conductor model. This unit generates synthetic and realistic brain signals, using realistic source models such as, but not limited to, NMM, which will be used to train the deep neural network architecture. The electromagnetic traces and recordings of these signals at scalp (or within the brain), i.e., EEG/MEG (or iEEG), will also be generated for these training datasets. These training data will be passed to the Network Training Unit and will also be saved in memory for further use. The Network Training Unit 1212 trains a deep neural network. The synthetic realistic data generated at the source and projected to the scalp, will be used to train the deep neural network architecture and the weights (and all relevant parameters of the proposed network architecture) are tuned based on these training datasets (and corresponding cross validation schemes, etc.) and saved in the memory to be ultimately passed to the Imaging Unit, where the underlying brain activities are estimated from real EEG/MEG/iEEG data recordings. Computing/Processing Unit 1204 processes the collected data. This unit performs some pre-processing on the data which includes filtering, removing noisy artefacts, extracting useful features from EEG/MEG/iEEG recordings to be passed to the Imaging Unit to delineate underlying brain activities from scalp measurements. The Computer Processing Unit 1204 may comprise a processor and a non-transitory, computer-readable storage medium storing instructions that, when executed by the processor, implement the method of the invention. The Imaging Unit 1206 performs source imaging using the trained neural network on collected electrophysiological data. The Imaging Unit 1206 performs the source imaging on the processed/raw EEG/MEG/iEEG recordings using the trained neural network architecture and weights loaded from the Storage Unit (trained in the Network Training Unit and saved in the Storage Unit), to provide estimates of underlying brain's activity, spatiotemporally. The results are passed to the Output Unit 1214, which displays the final estimates using computer graphics and on realistic brain structures so that physicians or researchers (or users) can better observe and interpret the results. The results are also saved in computer memory.

The invention disclosed herein accurately estimates the spatial and temporal information of the ictal sources, providing a high temporal correlation value of 0.95±0.07 in computer simulations, and of 0.81±0.14 in patient data analysis. The model can also provide high spatial specificity (98% in simulations and 96% in patient data analysis) with decent spatial sensitivity (84% in simulations and 44% in patient analysis), which means the model can provide accurate localization without having nuisance spurious activities extended outside the resection region. Multiple factors can influence the resection volume in practice, including the imaging and functional testing findings, the patient's treatment goals, the encountered individual anatomical reality during an operation, neurosurgeon's assessment of risks, the resection region is not necessary the ground truth for the ictal activities.

It is challenging to achieve both high sensitivity and high specificity, as there is a tradeoff between providing sparse or diffused solutions. The well-established conventional ESI methods are known to have high sensitivity but low specificity. DeepSIF has a low sensitivity compared to some diffused conventional methods (like sLORETA and LCMV), however, DeepSIF has a significantly higher specificity, leading to a higher geometric mean value compared to benchmark methods. On the other hand, the ictal imaging results from DeepSIF had a good overlap with the iEEG SOZ electrodes. As the SOZ LE is defined as average of distance of each SOZ to the closest reconstruction and the distance of each reconstructed region to the closest SOZ, a solution needs to be close to the SOZ electrode groups and be neither too diffused nor too focal to achieve a low LE value. DeepSIF reached a LE of 16.94±9.08 mm for all patients and 12.75±5.80 mm for seizure free patients, which are statistically significantly smaller than the benchmark methods.

Ictal imaging can more reliably estimate epileptogenic tissue compared to spike imaging. Several studies have demonstrated the advantage of ictal imaging over spike imaging. By comparing the DeepSIF ictal and spike imaging results, it can be seen that the ictal imaging is statistically significantly more accurate than the spike imaging in terms of sensitivity, specificity and localization error based on iEEG defined SOZ. When separating the patient groups into seizure free and non-seizure free groups, it can be observed that the performance difference is mainly caused by the multiple interictal spike types, while seizure sources are usually consistent and ipsilateral to the clinical ground truth. Previous studies have also observed that interictal spike clusters could be discordant with iEEG findings, and spike imaging would fail to accurately identify the EZ in these cases. Ictal imaging results become more critical in this case to resolve the inconsistency in spike imaging. Our ictal imaging approach can provide valuable information during the surgical evaluation process regarding the location of the SOZ and EZ, by providing an accurate ictal imaging result with high spatial specificity.

DeepSIF can provide robust extent, location, and temporal dynamics estimation for imaging ictal oscillations from scalp EEG in numerical experiments and real data analysis in 33 drug-resistant focal epilepsy patients. As a DL-based ESI method, it has the advantage of fast inference and no parameter tuning during the evaluation phase, while providing accurate and robust imaging results. The model's adaptability and reliability in handling diverse ictal patterns make it a promising tool for advancing noninvasive imaging of ictal activities in patients with epilepsy.

As stated above, the present invention is applicable to both EEG and MEG source imaging of brain activity. While most descriptions referred to EEG for simplicity of description, the methods disclosed above are applicable to either EEG, or MEG, or combination of EEG/MEG. Furthermore, while the detailed methods were disclosed to image brain sources from scalp EEG or MEG, the present invention is also applicable to image brain activity from recordings made in part or in full within the brain, such as intracranial EEG (iEEG) recordings, or a combination of iEEG and scalp EEG (or/and MEG) recordings.

While example results are shown for applications to epilepsy source localization and imaging, the present invention may also have applications to study brain normal functions to understand the mechanisms of brain functions, or to assist in diagnosis and management of other brain disorders, including various neurological and mental disorders.

One embodiment can be the application to brain computer interface, where the present invention can be used to efficiently perform ESI from noninvasive EEG or MEG to estimate the intent of a human subject for the purpose of communications or control of an external device or modulating the internal state of the subject for the purpose of rehabilitation or treating brain disorders.

1. A method comprising:
providing a forward source model of the brain;
causing the forward source model to generate training data comprising neural oscillations produced by the forward source model;
using the training data to train an inverse neural network model to perform an electrophysiological source imaging task to identify and localize regions generating the said neural oscillations in a brain.

2. The method of claim 1 wherein the neural oscillations are ictal oscillations in an epileptic brain.

3. The method of claim 1 wherein the forward source model comprises one or more neural mass models.

4. The method of claim 1 wherein a source space is modeled as a distribution of sources in the brain.

5. The method of claim 4 wherein the sources are current diploes.

6. The method of claim 4 wherein the inverse neural network learns the distribution of sources via the training data.

7. The method of claim 1 wherein the training data comprises a plurality of source-sensor signal pairs.

8. The method of claim 2 wherein the forward source model comprises a Jansen-Rit model modified to generate training data with various spatio-temporal-spectral features indicative of ictal oscillatory activity.

9. The method of claim 8 wherein the forward source model models different types of ictal oscillations.

10. The method of claim 8 wherein the modified Jansen-Rit model models signals comprising normal activity, sporadic spikes, sustained discharge of spikes, rhythmic activity, low voltage rapid activity and quasi-sinusoidal activity.

11. The method of claim 8 wherein the modified Jansen-Rit model is caused to produce ictal oscillations based on a selection of input parameters.

12. The method of claim 2 wherein the seizure-generating tissues are modelled as a source patch of brain tissue.

13. The method of claim 12 wherein the entire source patch shares a temporal waveform.

14. The method of claim 12 wherein the source patch is separated into a center segment and a neighboring segment, the center segment and neighboring segment having different temporal waveforms.

15. The method of claim 12 wherein source patches of differing sizes, shapes, locations, and temporal dynamics are generated as part of the training data.

16. The method of claim 1 wherein the inverse neural network model comprises a spatial module and a temporal module.

17. The method of claim 12 wherein the inverse neural network model is trained to model ictal neural oscillations based on an input of ictal EEG or MEG data.

18. The method of claim 1 wherein the inverse neural network model comprises:
a spatial filter that takes into account spatial information of the training data, the spatial information projecting sensor space measurements to source space signals in specific source regions in the brain; and
a temporal filter that takes into account temporal information of the training data, to estimate activity of the sources at given time in the output unit over a time interval.

19. A biophysically constrained deep neural network trained to image and localize source patched of ictal oscillations in a brain, the neural network being trained using a synthetic EEG, MEG or iEEG trace simulated by:
stimulating sources in a model of the brain; and
projecting source signals generated by the stimulations onto a model of a scalp to create the synthetic EEG or MEG or iEEG trace.

20. The deep neural network of claim 19 wherein the synthetic EEG, MEG or iEEG traces are modeled using a Jansen-Rit model modified to produce ictal oscillations.

21. The deep neural network of claim 19 wherein the deep neural network is trained to localize and image sources from ictal oscillations in an EEG, MEG or iEEG trace of an epilepsy patient.

22. A system for source imaging of electrical activity in a brain, the system comprising:
a physiological recording unit configured to record, at multiple locations, signals of brain electrical activity;
a computing/processing unit configured to:
process the recorded signals of brain electrical activity from the physiological recording unit;
simulate brain electrical activity using a realistic source model and electromagnetic signals on the sensor array;
train a neural network using the simulated sensor data corresponding to the source signals of simulated brain electrical activity;
a storage unit to store the trained neural networks and electromagnetic measurements in the sensor space;
an imaging unit to estimate brain source distributions given measurements in the sensor space using the trained neural network; and
an output unit to visualize spatial images or spatiotemporal signals of brain sources.
